(19) United States
(12) Patent Application Publication
(10) Pub. No.: US 2024/0404682 A1
(43) Pub. Date: Dec. 5, 2024

(54) Artificially Intelligent Systems, Methods and Media for Identification, Quantification and Correction of Defects in Health Care Services

(71) Applicant: Motive Medical Intelligence, San Francisco, CA (US)

(72) Inventors: Julie A. Scherer, Emerald Hills, CA (US); Richard S. Klasco, Englewood, CO (US); Nicolapagos A. Rains, Austin, TX (US); Larie H. Smoyer, Stevensville, MT (US); Charles M. Wikman, Minneapolis, MN (US); Jeanne Cohen, San Francisco, CA (US)

(21) Appl. No.: 18/611,501

(22) Filed: Mar. 20, 2024

Related U.S. Application Data
(63) Continuation-in-part of application No. 17/867,514, filed on Jul. 18, 2022, now Pat. No. 11,942,215.

(51) Int. Cl. G16H 40/20; G16H 20/00; G16H 50/20; G16H 70/20

(52) U.S. Cl. CPC G16H 40/20; G16H 20/00; G16H 50/20; G16H 70/20

(57) ABSTRACT

The disclosure pertains to a secure intelligent networked system for identifying and correcting a defect in a health care service and method for using the same. The system receives data regarding patient care, generally obtained from commercial insurance claims, customer claims or Medicare claims. The system operates on a plurality of nodes configured based on a set of metrics associated with the appropriateness of health care measures. The system may generate a determination regarding the appropriateness of the measure. The system may further produce a second determination denoting a physician's overall conformity with appropriate standards of practice. Finally, the system may generate a knowledge narrative indicating an appropriate action.

### cross-reference to related applications

The present continuation-in-part application claims the priority benefit of U.S. patent application Ser. No. 17/867,514 filed on Jul. 18, 2022 and titled “Systems and Methods for Identifying and Correcting Defects in Health Care Services,” the disclosure of which and all appendices are hereby incorporated by reference in their entireties.

### FIELD OF THE TECHNOLOGY

Embodiments of the present disclosure relate to the use of artificial intelligence in health care services.

### SUMMARY

Exemplary embodiments provide systems and methods for identifying and correcting defects in the cost, quality, and appropriateness of health care services.

Exemplary systems include: a secure intelligent networked system for processing data for a physician-directed health care service, such as a treatment plan. The data may be in the form of unstructured narrative text. Some exemplary systems may comprise a plurality of input and output nodes, as well as intermediary nodes configured based on a set of metrics associated with the appropriateness of care, others may not. The metrics may include information regarding the cost of health care services like those directed by the physician, or regarding the standard of care in the physician's specialty or region of practice, or other information related to the appropriateness of a health care service.

Subsequently, according to some exemplary embodiments, the input data are operationalized to produce a ranking of the appropriateness of the physician-directed health care service. A plurality of such rankings may be fed back into the system to determine overall appropriateness of health care services.

Some exemplary methods include the method of using the secure intelligent networked system for processing data for a physician-directed health care service. The method includes submitting such data with any level of operable structure, including unstructured narrative text. The method may further comprise the use of a plurality of input and output nodes, as well as intermediary nodes configured based on a set of metrics associated with the appropriateness of care, other methods may not. The metrics may include information regarding the cost of health care services like those directed by the physician, or regarding the standard of care in the physician's specialty or region of practice, or other information related to the appropriateness of a health care service.

Subsequently, according to some exemplary methods, the input data are operationalized to produce a ranking of the appropriateness of the physician-directed health care service. This ranking may be compared to similar rankings to determine whether the service is appropriate considering expert opinion or the relevant standard of care. A plurality of such rankings may be used as a benchmark for appropriateness of health care services. Individually, the ranking may be used to alter or validate the decision to implement the physician-directed healthcare service.

According to some exemplary embodiments, the system produces an output which in turn produces an outcome, which in turn produces an input. In some embodiments, the output may become the input.

### BRIEF DESCRIPTION OF THE DRAWINGS

Exemplary embodiments are illustrated by way of example and not limitation in the figures of the accompanying drawings, in which like references indicate similar elements.

FIG. 1 is a flowchart of an example method of the present disclosure.

FIG. 2 diagrammatically illustrates an example method of receiving input data in various

levels of operable structure.

FIG. 3 is an example method of presenting the output or outputs of the system and

method described herein.

FIGS. 4A, 4B, and 4C depict a flowchart related to adjustment of weighted values as used in the present disclosure.

FIG. 5 is a further example method of presenting and contextualizing the output or outputs of the system and method described herein.

FIG. 6 is a further example method of presenting and contextualizing the output or outputs of the system and method described herein.

FIG. 7 is an exemplary embodiment of the secure intelligent network on which the

disclosure may be implemented.

FIG. 8 is an exemplary embodiment of a deep neural network, as may be used in the present disclosure.

FIG. 9 shows an exemplary large language model.

### DETAILED DESCRIPTION

The elements identified throughout are exemplary and may include various alternatives, equivalents, or derivations thereof. Various combinations of hardware, software, and computer-executable instructions may be utilized. Program modules and engines may include routines, programs, objects, components, and data structures that effectuate the performance of a particular task when executed by a processor. Computer-executable instructions and associated data structures stored in a computer-readable storage medium represent examples of programming means for executing the steps of the methods and/or implementing particular system configurations disclosed herein.

The terminology used herein is for the purpose of describing particular embodiments only and is not intended to be limiting of the technology. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that like or analogous elements and/or components, referred to herein, may be identified throughout the drawings with like reference characters. It will be further understood that several of the figures are merely schematic representations of the present disclosure. As such, some of the components may have been distorted from their actual scale for pictorial clarity.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments.

Depending on the context of discussion herein, a singular term may include its plural forms and a plural term may include its singular form. Similarly, a hyphenated term may be occasionally interchangeably used with its non-hyphenated version, a capitalized entry may be interchangeably used with its non-capitalized version, and an italicized term may be interchangeably used with its non-italicized version. Such occasional interchangeable uses shall not be considered inconsistent with each other.

### Identification of Defects

The present disclosure describes artificially intelligent systems, methods and media for the rapid identification of defects in health care services, and further, the correction of defects.

The term “defect” refers to decisions or behaviors that result in low-value or inappropriate care, or waste in health care delivery systems. A health care system that minimizes defects can more effectively maximize safety and quality, as well as minimize cost and waste.

A common type of defect is inappropriate care, which deviates from evidence-based practices and can expend limited resources.

Appropriateness measures are measures designed to reduce the occurrence of defects in health care services. Appropriateness measures include measures that discourage deviation from evidence-based practices or that reduce waste.

Appropriateness measures may reduce the incidence of overuse or underuse of a health care action. By way of example, appropriateness measures would include reducing the incidence of mammography screening underuse in eligible women, physical therapy underuse before lumbar surgery, or nuclear imaging overuse in stress testing.

Appropriateness measures can be tools for making visible and correcting defects.

Appropriateness measures are built on a conjoint analysis of cost, quality, and potential for harm. This foundation aligns the interests of payers, providers, and patients.

Efforts that individually optimize cost, quality, and safety frequently fail to acknowledge the implicit tradeoffs that appropriateness demands. Costs must be constrained but not at the expense of quality; quality is desirable but not at all costs; and safety concerns cannot be allowed to thwart potentially lifesaving treatments that entail risk.

### Artificially Intelligent Clinical Decision Support

Artificially Intelligent Clinical Decision Support provides patients, health care providers, payors, and other individuals with knowledge and person-specific information that is intelligently filtered and presented when needed or requested. Artificially Intelligent Clinical Decision Support can significantly improve quality, safety, efficiency, and effectiveness of health care services.

Artificially Intelligent Clinical Decision Support requires computable biomedical knowledge, person-specific data, and a reasoning or inferencing mechanism that will generate useful information based on the knowledge and data. An important goal is to receive such information before, during and/or after care is delivered.

Traditionally, practice guidelines and quality officers were relied upon to determine the appropriateness of a health care action. However, reliance on practice guidelines and quality officers can be inefficient due to time delays. Such reliance is also frequently not scalable or extensible and is often seen as subjective rather than quantifiable.

Some of the artificially intelligent systems, methods and media disclosed herein allow appropriateness measures to be constructed as machine-executable knowledge objects, allowing them to be run effectively against big data. In this way, appropriateness measures may be quantifiable, scalable, extensible, and more readily accessible by end users.

In some of the artificially intelligent systems, methods and media disclosed herein, the appropriateness of a health care action may be determined based on evidentiary support such as medical journals, health studies, clinical guidelines, standards bodies, or subject matter expert panels.

Moreover, the input received from such evidentiary support may be quantified, operationalized, and processed by way of an evidence engine and/or artificial intelligence implemented on a secure intelligent communications network. The secure intelligent communications network may be implemented on a system comprising a processor to execute instructions stored in memory and performing asynchronous processing with a computing device. Some or all of the activities occur over one or more network/communication links and may occur in a cloud computing system or edge computing system.

The secure intelligent communications network may further be implemented on a deep-learning environment such as a deep neural network consisting of an input node, a plurality of intermediary nodes, and an output node, each node having weights, biases, functions, and thresholds that are implemented and tuned for optimally useful results.

In some of these embodiments, each node connects to another node and has an associated weight and threshold. If the output of any individual node is above a specified threshold value, that node will transmit data to the next node within the network. If the output of any individual node is below a specified threshold value, no data will be transmitted to the next node.

Neural networks and/or Large Language Models (“LLMs”) in some of these embodiments rely on training data to develop and improve accuracy over time. Once finely tuned for precision, the networks are powerful tools that allow for classification, clustering, and processing large amounts of data at high speeds.

Further, in some of these embodiments, once an input layer is established, weights are assigned. Such weights help determine the importance of a variable, with larger weights contributing in greater proportion to the output compared to smaller weights. The inputs are multiplied by their respective weights and added together, after which an activation function is applied to the result. If the output exceeds a given threshold, the node is activated, and will transmit data to the next node in the network.

While some embodiments are feedforward, with data flowing only in the direction of input to output, it should be noted that further embodiments may be trained through backpropagation. In such embodiments, data may be transmitted from the output node toward the input node, allowing for calculation and attribution of error associated with each node. Parameters may be adjusted accordingly.

Further, according to some exemplary embodiments, the system produces an output which in turn produces an outcome, which in turn produces an input. In some embodiments, the output may become the input.

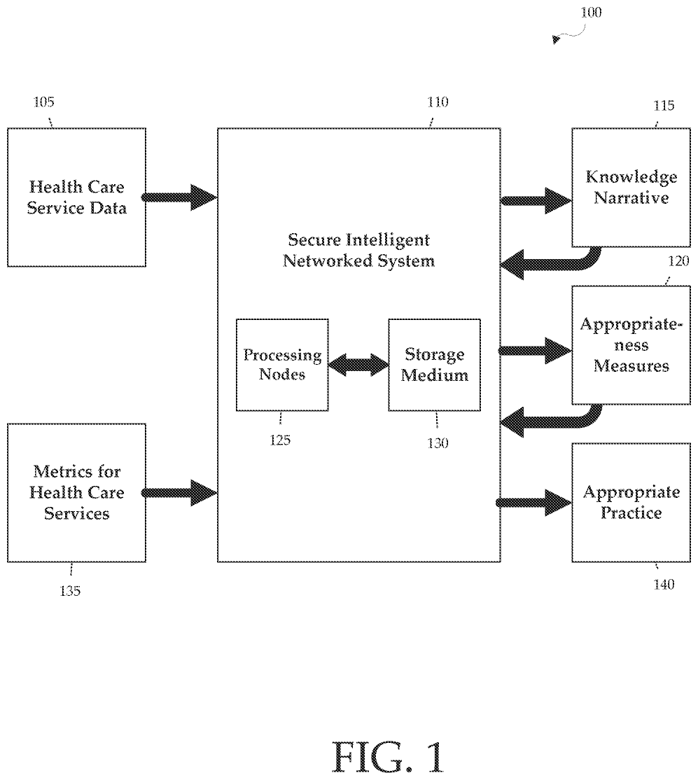

FIG. 1 shows an exemplary implementation of the disclosed systems and methods. Within the secure intelligent networked system 110, clinical input may be assigned operative values with various levels of structure, from Boolean operators to coded information interpretable by Artificially Intelligent Clinical Decision Support systems.

The clinical input may include patient health care data received from commercial insurance claims, customer claims, or Medicare claims, or other physician-submitted data sources 105.

The secure intelligent networked system may be configured to perform a series of operations on the received clinical input. Such configuration may include additional inputs from subject matter panels or clinical data 135, or may include weighted values across a plurality of nodes, the weights being associated with values pertaining to cost, quality, and/or appropriateness of care.

The secure intelligent networked system may generate a number of useful outputs from these operations, including, for example, a knowledge narrative 115. Such knowledge narrative may include a plain text recommended health care action.

By way of example, a knowledge narrative may indicate “75 milligrams of acetaminophen.”

The knowledge narrative may be distilled with further iterations to account for nonviable knowledge narratives.

By way of example, a knowledge narrative such as “75 milligrams of acetaminophen” may not be viable for some allergies or comorbidities. In such cases, the set of metrics may be adjusted to account for such nonviability.

The secure intelligent networked system may further produce an additional output denoting a physician's Appropriateness Measures Score 120.

The Appropriateness Measures score refers broadly to the of ranking a physician's use of appropriate and inappropriate actions regarding a specific type of health care service.

In one embodiment, the method first takes the percentage of cases handled by the physician with inappropriate care actions out of cases handled by the physician that qualify for the care action.

As appropriate, cases may be excluded from the overall number of qualifying cases if complicating factors, increased risk, or urgent or emergent circumstances are present.

By way of example, an Appropriateness Measures Score may be determined for cardiovascular stress testing by evaluating a physician's number of inappropriate stress tests out of the number of all stress test cases.

Such inappropriate stress tests may include, by way of example, overuse of nuclear stress testing, which in some circumstances may impose significant patient cost burden, and can be avoided by ordering alternative diagnostic testing.

Numerator cases in this example would include stress testing with nuclear imaging that occurred within 30 days of an evaluation and management visit to a cardiologist. Denominator cases would include stress testing that occurred within 30 days of a cardiologist evaluation and management visit. Excluded cases would include inpatients, outpatients with symptoms of Acute Coronary Syndrome, or patients who had a cardiac-related emergency department visit during the 30-day period.

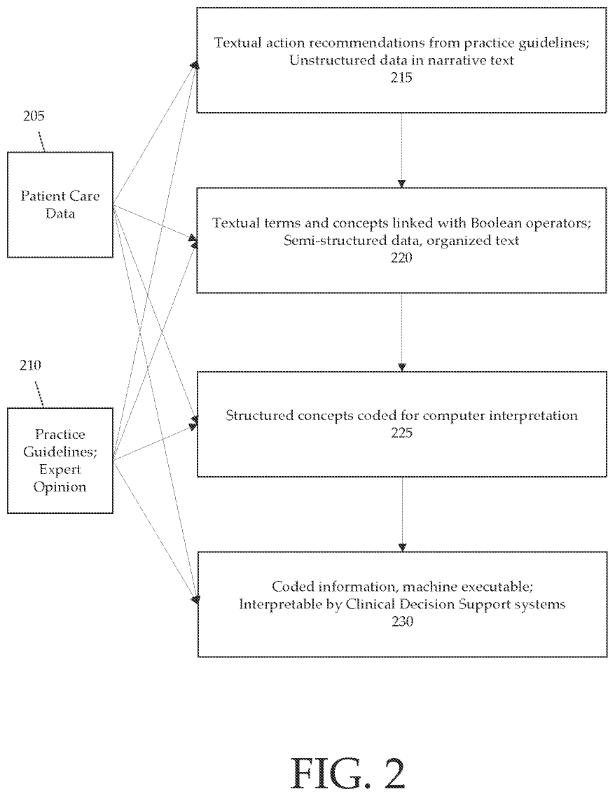

FIG. 2 diagrammatically illustrates an exemplary implementation of an artificially intelligent evidence engine on the secure intelligent networked system. Using a large language model (LLM), a multilayered knowledge schema may be applied to process the data within the secure intelligent networked system. The LLM could generate the knowledge outputs of narrative, semi-structured, structured, and coded. The LLM could be trained to produce computable biomedical knowledge outputs using representative training sets of healthcare narrative text, semi-structure knowledge representation, structured healthcare concepts coded for computer interpretation and machine executable representations of healthcare services logic. Training techniques for the LLM could include a mix of supervised learning, unsupervised learning, reinforcement learning, defect identification and correction using iterative training algorithms, and stochastic learning models. The combination of training techniques applied at each level could be determined and refined to generate the output.

The evidence engine may be operated based on and iteratively to produce measures represented in JavaScript Object Notation (JSON) data interchange format and in the Denominator, Exclusion, Attribution, Numerator (DEAN) structure to facilitate logical query flow. Concepts may be expressed in comprehensive value sets. Minimum denominators may be established using standard techniques, such as Bühlmann-Straub credibility modeling. In some embodiments, training sets of DEAN structures, value sets and minimum denominator values could be applied to the LLM to generate the measure JSON representation.

In some embodiments, the multilayered knowledge schema may be adapted from Boxwala et al., “A Multi-Layered Framework for Disseminating Knowledge for Computer-Based Decision Support.”

In such embodiments, the data received may have any level of structure, including unstructured narrative format 215. The narrative text may include patient care data 205 received from physician-submitted commercial insurance claims, customer claims, or Medicare claims. The narrative text may also include textual recommendations from practice guidelines or expert opinion and may be curated by subject matter expert panels 210. In some embodiments, an LLM could be trained using structured and unstructured formats of patient care data.

The received data may be semi-structured 220, with terms and concepts linked to Boolean operators. Logic operations may then produce recommendations regarding the interventions that are possible in a specified clinical scenario. In some embodiments, training sets of semi-structured representations of healthcare measures criteria and Boolean operators could be used to train an LLM.

The received data may be further specified with sufficient structure as to be coded and interpretable by a computer 225. In this structured state, the text formally defines all data elements and logic required to use a computer based Artificially Intelligent Clinical Decision Support system. In some embodiments, training sets of healthcare measures logic and concepts coded for computer interpretation could be used to train an LLM.

The received data may be further structured in machine-executable format for use in various Artificially Intelligent Clinical Decision Support systems 230. In some embodiments, the LLM could be trained using healthcare analytics programs and code libraries. Clinical knowledge may then be implemented within a specified setting and workflow.

### Range of Better Practice

The Range of Better Practice refers to the limits of appropriate measures, where an Appropriateness Measures score may exceed an upper threshold in the case of overuse of an action, or lower threshold in the case of underuse of an action.

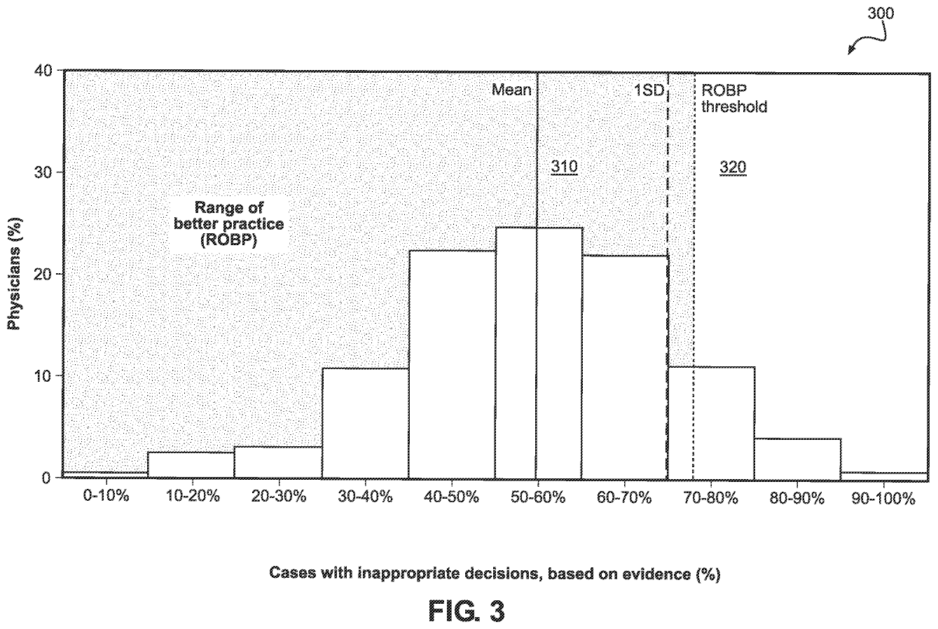

FIG. 3 illustrates an exemplary Appropriateness measure 310 in context with the Range of Better Practice 320. The Range of Better Practice may be determined first by statistical analysis of claims data such as identification of outliers on a Gaussian curve. Algorithms may be implemented within the network to adjust for factors that may not be apparent from the claims data.

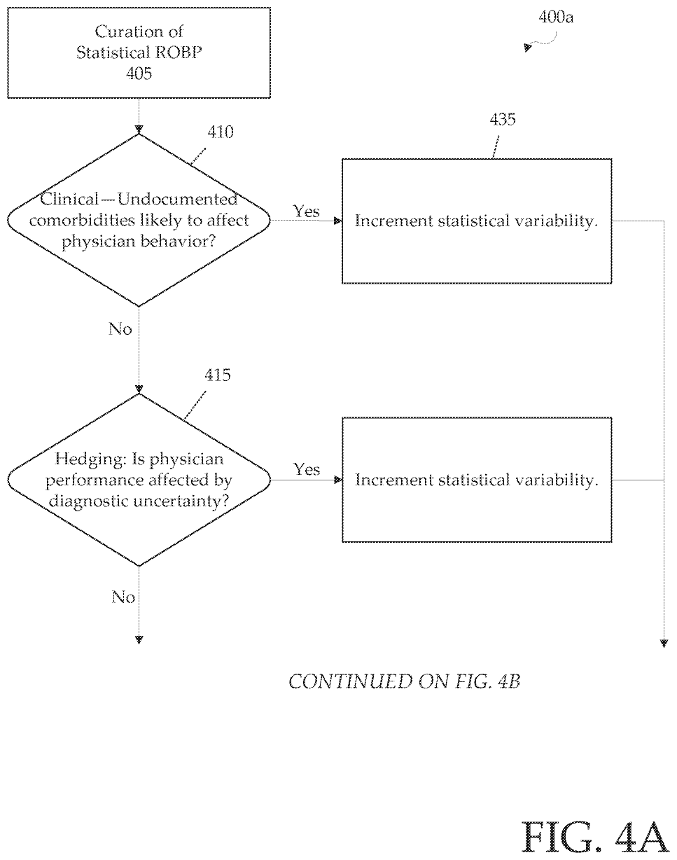

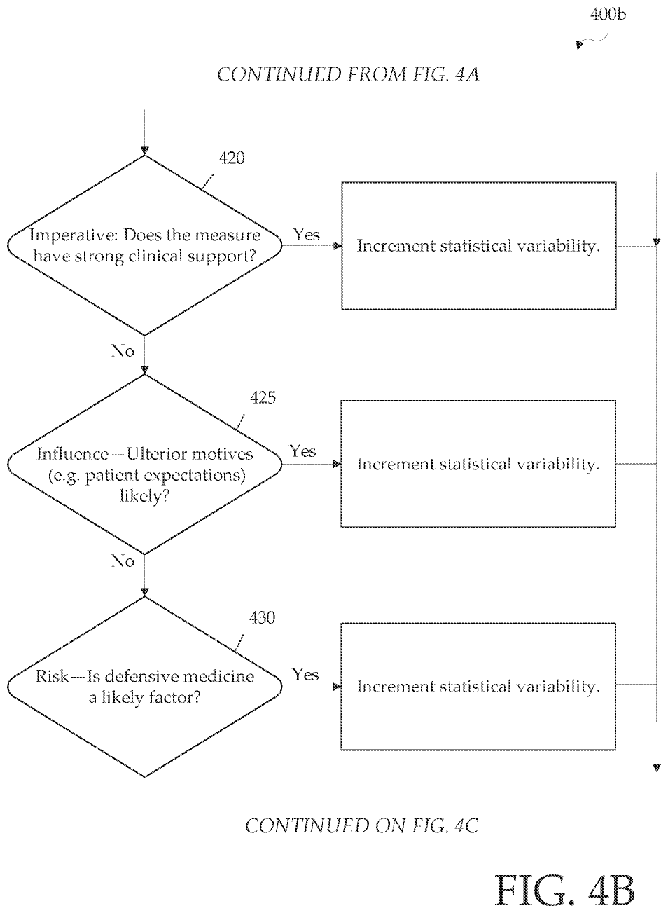

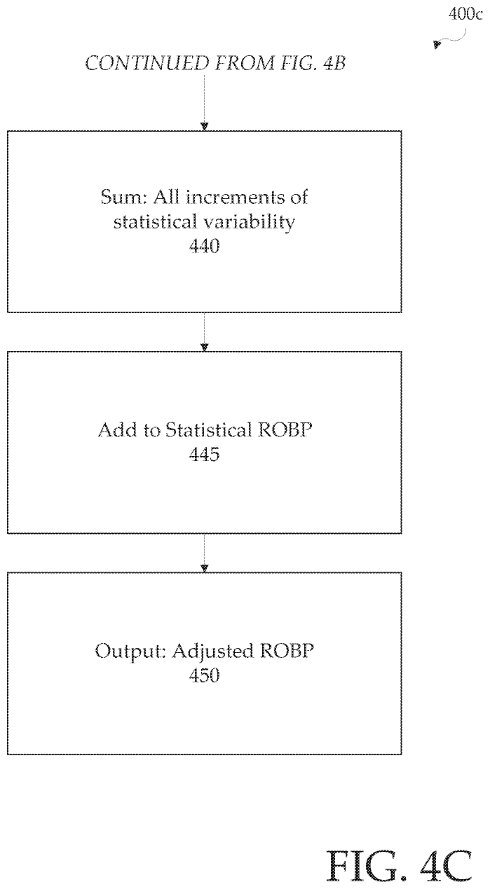

FIGS. 4A, 4B, and 4C illustrate an exemplary algorithm that may be used to adjust for factors that may be lacking in claims data 400.

In one embodiment of the algorithm, such factors may include undocumented comorbidities 410; hedging in the face of diagnostic uncertainty 415; strength of clinical support 420; ulterior factors such as patient expectations 425; or defensive practice to reduce any risk of malpractice liability 430.

Each such factor may add one increment of variability 435. The sum of such increments can be used to adjust the Range of Better Practices as appropriate 440.

In the example of nuclear stress testing, clear overuse of nuclear stress testing was found where more than 65% of cardiovascular stress testing implemented were unnecessary, or otherwise inappropriate, nuclear stress tests.

### Appropriate Practice

The disclosed methods may further allow for a cumulative Appropriate Practice Score to reflect a physician's performance across multiple measures or practice areas.

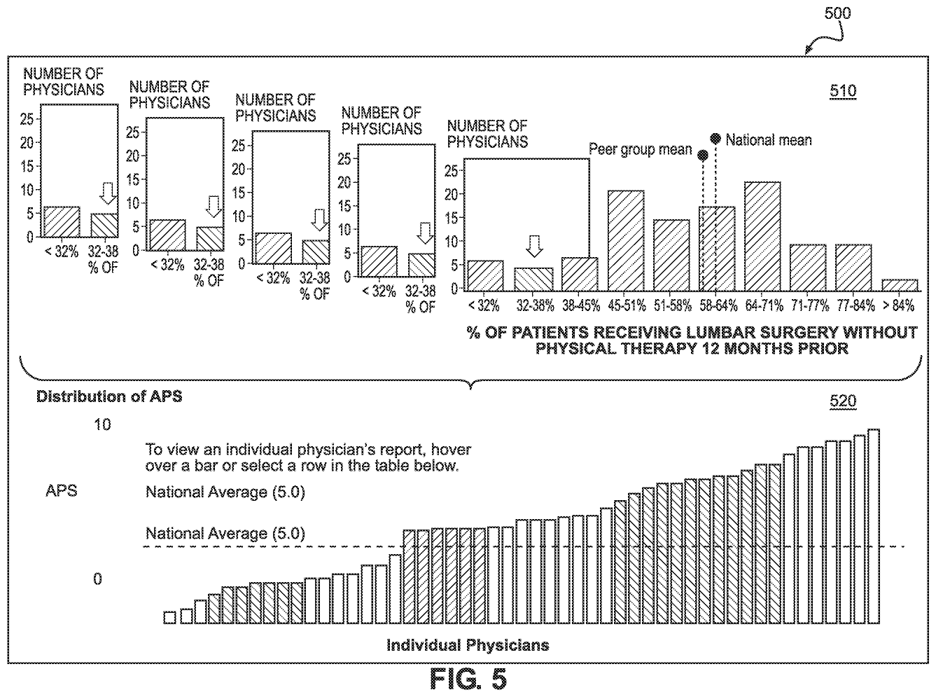

FIG. 5 is an exemplary method of determining and displaying the Appropriate Practice Score. Individual Appropriateness Measures scores, in context with the Range of Better Practice, are accumulated 510 and reflected in a distribution of Appropriate Practice Scores for a plurality of physicians 520.

Appropriateness Measures scores may be weighted more heavily in the overall Appropriate Practice Score when the measures are more costly, more harmful, or practiced more frequently by an individual physician.

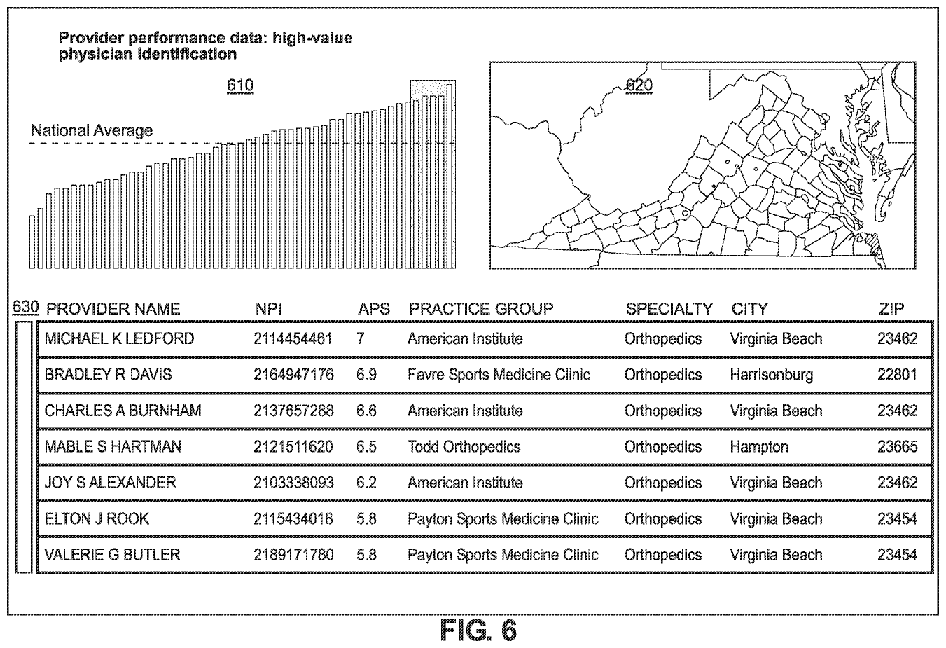

FIG. 6 shows an exemplary method of displaying the output for Appropriate Practice Scores. Appropriate Practice Scores may be compared and evaluated with a national average 610, or may be searched by region 620. Physician rankings by region or practice area 630 may also be displayed.

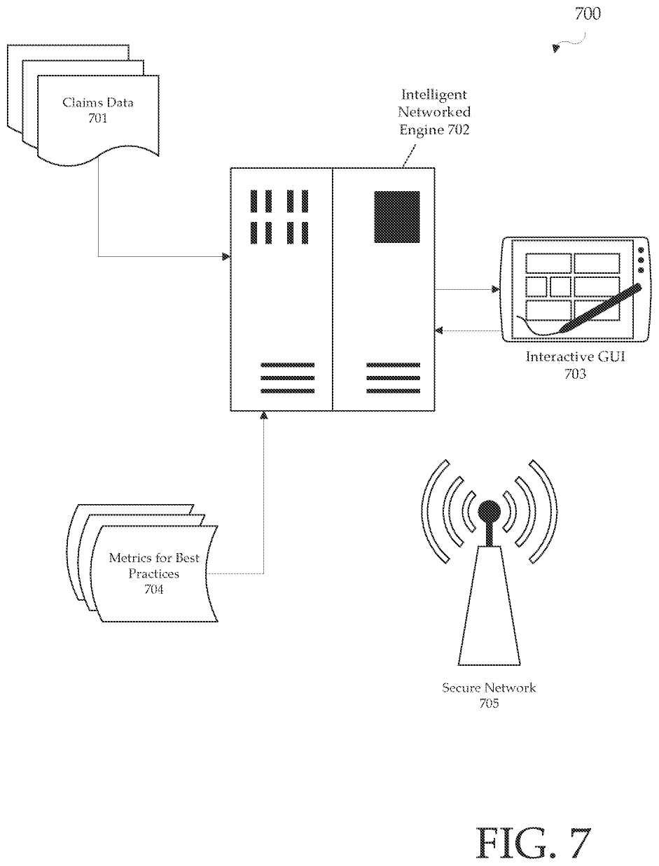

FIG. 7 shows an exemplary embodiment of the secure intelligent network on which the disclosure may be implemented. Physician-submitted claims data 701 may be received by the intelligent networked engine 702, which may or may not be comprised of a plurality of nodes operating according to a set of metrics 704. The outputs described herein may be displayed on an interactive graphic user interface 703 which may return further data to adjust values stored within the intelligent networked engine, or which may supplement the claims data as initially submitted. Such supplementation may modify or validate any output generated by the intelligent networked engine in accordance with best practice based on unique or specific circumstances.

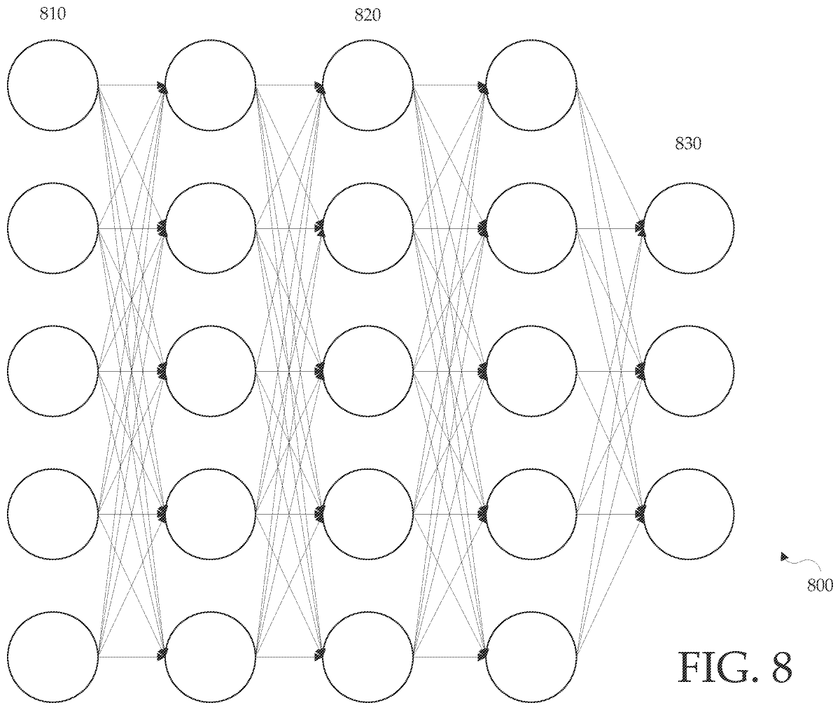

FIG. 8 shows an exemplary deep neural network which may be used in the implementation. The exemplary embodiment here comprises an input node 810, a plurality of intermediary nodes 820, and an output node 830, each node having weights, biases, functions, and thresholds that are implemented and tuned for optimally useful results.

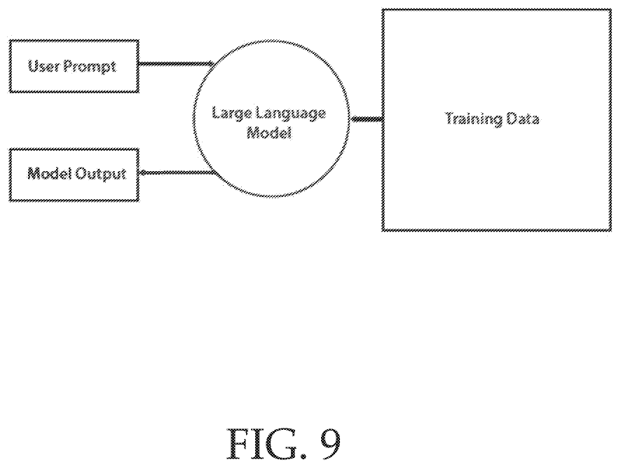

FIG. 9 shows an exemplary large language model. Shown in FIG. 9 is a user prompt, a large language model, training data, and a model output. A user prompt in a large language model (LLM) is a piece of text that is used to guide the LLM to generate a desired model output. The prompt can be used to specify the type of model output that the LLM should generate, as well as the style and tone of the output. The quality of the model output generated by an LLM is heavily influenced by the quality of the prompt. A well-crafted prompt will help the LLM to generate output that is more relevant, accurate, and creative.

An LLM is a type of artificial intelligence (AI) model that is trained on a massive amount of text data. This data can be text from books, articles, websites, or any other source of text. The LLM learns the patterns and structure of the text data, and it can then use this knowledge to generate new text, translate languages, write different kinds of creative content, and answer questions in an informative way. According to some exemplary embodiments, an LLM may be trained on medical journals, books, online resources, semi-structured, and structured knowledge representations, coding libraries and programs.

For example, the text, semi-structured, structured, and machine executable data employed in some of the exemplary embodiments described herein may include:

Medical Journals:

Journal of the American Medical Association (JAMA)-Publishes research articles, reviews, and guidelines on various medical procedures and their appropriateness.

New England Journal of Medicine (NEJM)-Features clinical research and articles discussing the effectiveness and appropriateness of medical interventions.

Annals of Internal Medicine-Publishes studies, guidelines, and reviews on diagnostic and therapeutic procedures.

Journal of Nuclear Cardiology-Focuses specifically on nuclear imaging techniques used in cardiology, including stress testing.

European Journal of Nuclear Medicine and Molecular Imaging-Covers research on nuclear imaging techniques and their clinical applications.

Books:

Clinical Practice Guidelines for Nuclear Medicine Practice by Society of Nuclear Medicine and Molecular Imaging (SNMMI)-Provides guidelines and recommendations for various nuclear medicine procedures, including stress testing.

Cardiac Nuclear Medicine by Gary V. Heller-Offers an in-depth understanding of cardiac nuclear imaging techniques, including stress testing protocols and interpretation.

Textbook of Clinical Nuclear Medicine edited by Henry N. Wagner Jr., M.D. et al.-Covers the principles, techniques, and clinical applications of nuclear medicine imaging, including stress testing.

Essentials of Nuclear Medicine Imaging by Fred A. Mettler Jr.—Provides a comprehensive overview of nuclear medicine imaging modalities, including stress testing and its clinical relevance.

ACC/AHA/ASE 2003 Guideline Update for the Clinical Application of

Echocardiography—Published by the American College of Cardiology (ACC), American Heart Association (AHA), and American Society of Echocardiography (ASE), this guideline offers recommendations for appropriate use criteria for echocardiography, including stress echocardiography.

Online Resources:

American College of Cardiology (ACC) Guidelines-Provides guidelines and appropriate use criteria for various cardiac imaging modalities, including stress testing.

Society of Nuclear Medicine and Molecular Imaging (SNMMI) Guidelines-Offers guidelines and recommendations for nuclear medicine procedures, including stress testing.

UpToDate—Online medical resource offering evidence-based information on various medical topics, including appropriateness criteria for diagnostic procedures.

Healthcare Measure Specifications

National Committee on Quality Assurance Healthcare Effectiveness Data and

Information Set (HEDIS) measure specifications-Provides textual specifications for measure logic and criteria to evaluate healthcare insurance performance.

Centers for Medicare and Medicaid Electronic Clinical Quality Measures (eCQM)—Provides textual specifications for quality evaluation of healthcare systems.

Semi-structured and Structured Representations.

Knowledge representations of healthcare measures, such as Denominator, Exclusion,

Attribution, Numerator (DEAN), the Centers for Medicare and Medicaid Electronic Clinical Quality Measures (eCQM) and Healthcare Effectiveness Data and Information Set (HEDIS)

Value Set Authority Center (VSAC) Value sets—Provides specifications for a set of codes defining patient attributes, healthcare venues, financial information or medical concepts drawn from one of more medical code systems, for example.

Medical claims and clinical data.

Fast Healthcare Interoperable Resource (FHIR), Clinical document Architecture (CDA), and Quality Data Model (QDM)—Clinical and claims data standards for storage, exchange and processing from Health Level Seven International.

Coding Libraries and Programs.

Healthcare data analytics programs and code samples using computer languages, such a SAS, SQL, Apache Spark, python, and R.

Clinical decision support, healthcare analytics, healthcare measures and other computable biomedical knowledge coded in the Clinical Quality Language.

Risk-adjusted healthcare measures in SQL, Python and R.

As previously mentioned, LLMs are advanced artificial intelligence algorithms trained on massive amounts of text data for the purposes of content generation, summarization, translation, classification, sentiment analysis; large amounts of code and programs for the purpose of code generation, and so much more. For example, the LLM could be trained on exemplars such as medical claims, clinical data, coding libraries and/or programs. Smaller datasets are composed of tens of millions of parameters, while larger sets extend into hundreds of billions of data points. Depending on the purpose of the LLM, the training data will vary.

Transformer architecture is the backbone of the transformer models like GPT and many other prominent LLMs. The transformer architecture is a neural network architecture that allows for parallel processing and is used by large language models to process data and generate contextually relevant responses. It consists of a series of layers, with each layer consisting of parallel processing components called attention mechanisms and feedforward networks. The attention mechanisms weigh the importance of each word, using statistical models to learn the relationships between words and their meanings. This allows LLMs to process sequences in parallel and generate contextually relevant responses.

LLMs and neural networks can be combined to work together. In some exemplary embodiments, this may be done by using the LLM to generate a set of features that are then fed into the neural network. The neural network can then use these features to make predictions or classifications. For example, in natural language processing, LLMs can be used to generate text features that are then fed into neural networks for tasks such as sentiment analysis, machine translation, and question answering.

The training of AI includes:

Supervised learning: In supervised learning, the AI is trained on a set of labeled data.

Unsupervised learning: In unsupervised learning, the AI is trained on a set of unlabeled data.

Reinforcement learning: In reinforcement learning, the AI is rewarded for identifying an item correctly. Over time, the AI consistently improves.

The specific approach that is used will depend on the specific needs of the application. For example, if the goal is to identify changes as soon as possible, then supervised learning may be a good option. However, if the goal is to understand the nuances of an item, then unsupervised learning or reinforcement learning may be a better option. In addition to the type of learning, the training of AI also depends on the size and quality of the data set. A larger data set will typically lead to better performance, but it may also take longer to train the AI. The quality of the data set is also important, as it should be representative of the types of documents that the AI will be used to analyze.

The AI can be trained with an expanded training set using stochastic learning with backpropagation which is a type of machine learning algorithm that uses the gradient of a mathematical loss function to adjust the weights of an AI network. Unfortunately, the introduction of an expanded training set tends to increase false positives when classifying data. Accordingly, a second feature may be the minimization of these false positives by performing an iterative training algorithm, in which the AI network is retrained with an updated training set including the false positives.

1. An intelligent secure networked system for identifying and correcting a defect in a health care service, the system comprising:
a computer processor for processing data;
a storage medium communicatively coupled to the computer processor, the storage medium storing data;
a secure intelligent network communicatively coupled to the computer processor and the storage medium, the secure intelligent network having a deep neural network, the deep neural network trained by an evidence engine with evidentiary support including medical journals, health studies, clinical guidelines, or standards bodies, the deep neural network configured to:
receive a set of data comprising physician-directed health care service data for a previous stress test as coded and unstructured narrative text, and health care service data as a health care service is being delivered, and configured to adjust for a factor lacking in claims data including undocumented comorbidities, hedging in diagnostic uncertainty, strength of clinical support, ulterior motives, defensive medicine, a presence for each factor equating to incremental statistical variability that is calculated to a sum, added to a statistical range of better practice and results in an adjusted range of better practice;
receive a set of metrics associated with appropriateness of a stress test;
have a weight, bias and threshold directing an analysis by the deep neural network on physician-directed health care service data for a stress test;
to generate a first output comprising a knowledge narrative representing a plain-text description of an appropriateness measure for the stress test and a range of better practice comprising limits of the appropriateness measure, where an appropriateness measures score exceeds an upper limit in a case of overuse of a service, or is below a lower limit in a case of underuse of a service that results from operation of the neural network on the input elements;
generate a second output that comprises a rate of inappropriateness of the stress test, the inappropriateness having a numerator representing a number of stress tests with nuclear imaging that occurred within 30 days of an evaluation and management visit to a cardiologist and having a denominator representing stress testing that occurred within 30 days of an evaluation and management visit to a cardiologist, excluding cases with inpatients, outpatients with symptoms of acute coronary syndrome or patients who had a cardiac-related emergency department visit within a thirty-day period;
have a dynamic feedback communicatively coupling the knowledge narrative and range of better practice node and the rate of inappropriateness of the stress test for the specific health care service for continuous learning of the deep neural network;
generate an appropriateness measures score for cardiovascular stress testing;
and generate a cumulative appropriateness practice score to reflect a physician's performance across multiple measures or practice areas.

2. The system of claim 1, further comprising the physician-directed healthcare service data that includes data received from a physician-submitted insurance claim.

3. The system of claim 1, further comprising the physician-directed healthcare service data that includes a diagnosis or a treatment plan.

4. The system of claim 1, further comprising a set of metrics including data regarding a medical standard of care.

5. The system of claim 4, further comprising the set of metrics including data regarding a cost of a medical service.

6. The system of claim 4, further comprising the set of metrics including data received from a commercial claim.

7. The system of claim 4, further comprising the set of metrics including data received from a customer claim.

8. The system of claim 4, further comprising the set of metrics including data received from a Medicare claim.

9. The system of claim 1, further comprising the second output node generating an output that comprises a plain text recommended action for a specific patient.

10. A method for identifying and correcting a defect in a healthcare service, comprising:
training a deep neural network by an evidence engine, the neural network trained with evidentiary support including medical journals, health studies, clinical guidelines, or standards bodies the deep neural network:
receiving by a secure intelligent networked engine having the deep neural network, a set of data comprising physician-directed health care service data for a previous stress test as coded and unstructured narrative text, and health care service data as a health care service is being delivered, the deep neural network configured to adjust for a factor lacking in claims data including undocumented comorbidities, hedging in diagnostic uncertainty, strength of clinical support, ulterior motives, defensive medicine, a presence for each factor equating to incremental statistical variability that is calculated to a sum, added to a statistical range of better practice and results in an adjusted range of better practice;
receiving, by the secure intelligent networked engine having the deep neural network, a set of metrics associated with appropriateness of a stress test;
configuring the deep neural network to have a weight, bias and threshold directing an analysis by the deep neural network on the physician-directed health care service data for a stress test;
generating a knowledge narrative representing a plain text description of an appropriateness measure for the stress test and a range of better practice comprising limits of the appropriateness measure, where an appropriateness measures score exceeds an upper limit in a case of overuse of a service, or is below a lower limit in a case of underuse of a service that results from operation of the neural network on the input elements;
generating a rate of inappropriateness of the stress test, the inappropriateness having a numerator representing a number of stress tests with nuclear imaging that occurred within 30 days of an evaluation and management visit to a cardiologist and having a denominator representing stress testing that occurred within 30 days of an evaluation and management visit to a cardiologist, excluding cases with inpatients, outpatients with symptoms of acute coronary syndrome or patients who had a cardiac-related emergency department visit within a thirty day period;
a dynamic feedback communicatively coupling the knowledge narrative and range of better practice node and the rate of inappropriateness of the stress test for the specific health care service for continuous learning of the deep neural network;
generating an appropriateness measures score for cardiovascular stress testing;
and generating a cumulative appropriateness practice score to reflect a physician's performance across multiple measures or practice areas.

11. The method of claim 10, further comprising the physician-directed healthcare service data including data received from a physician-submitted insurance claim.

12. The method of claim 10, further comprising the physician-directed healthcare service data including a diagnosis or treatment plan.

13. The method of claim 10, further comprising input for a set of metrics that is received from published guidelines, medical journals, standards organizations, or expert opinion.

14. The method of claim 13, further comprising the set of metrics including data regarding a medical standard of care.

15. The method of claim 13, further comprising the set of metrics that including data regarding a cost of a medical service.

16. The method of claim 13, further comprising the set of metrics including data received from a commercial claim.

17. The method of claim 13, further comprising the set of metrics including data received from a customer claim.

18. The method of claim 13, further comprising the set of metrics including data received from a Medicare claim.

19. The method of claim 10, further comprising generating an output including a plain text recommended action for a specific patient.

20. A non-transitory computer-readable storage medium having embodied thereon instructions, which when executed by a processor, perform steps of a method, the method comprising:
training a deep neural network, the deep neural network trained by an evidence engine with evidentiary support including medical journals, health studies, clinical guidelines, or standards bodies; the deep neural network:
receiving physician-directed healthcare service data for a previous stress test as coded and unstructured narrative text, the deep neural configured to adjust for a factor lacking in claims data including undocumented comorbidities, hedging in diagnostic uncertainty, strength of clinical support, ulterior motives, defensive medicine, a presence for each factor equating to incremental statistical variability that is calculated to a sum, added to a statistical range of better practice and results in an adjusted range of better practice;
receiving a set of metrics associated with appropriateness of a stress test;
configuring the deep neural network to have a weight, bias and threshold directing an analysis of the deep neural network on physician-directed health care service data for a stress test;
generating, by the secure intelligent networked engine having the deep neural network, a knowledge narrative representing a plain-text description of an appropriateness measure for the stress test and a range of better practice comprising limits of the appropriateness measure, where an appropriateness measures score exceeds an upper limit in a case of overuse of a service, or is below a lower limit in a case of underuse of a service that results from operation of the deep neural network on the input elements;
generating, by the secure intelligent networked engine having the deep neural network, a rate of inappropriateness of the stress test, the inappropriateness having a numerator representing a number of stress tests with nuclear imaging that occurred within 30 days of an evaluation and management visit to a cardiologist and having a denominator representing stress testing that occurred within 30 days of an evaluation and management visit to a cardiologist, excluding cases with inpatients, outpatients with symptoms of acute coronary syndrome or patients who had a cardiac-related emergency department visit within a thirty day period;
a dynamic feedback communicatively coupling the knowledge narrative and range of better practice and the rate of inappropriateness of the stress test for the specific health care service for continuous learning of the deep neural network;
generating an appropriateness measures score for cardiovascular stress testing;
and generating a cumulative appropriateness practice score to reflect a physician's performance across multiple measures or practice areas.

21. The system of claim 1, further comprising the evidence engine configured with:
textual action recommendations from practice guidelines;
unstructured data in narrative text;
textual terms and concepts linked with Boolean operators;
semi-structured data;
organized text;
structured concepts coded for computer interpretation; and
coded information, machine executable, interpretable by clinical decision support systems.
